(19) United States
(12) Patent Application Publication
(10) Pub. No.: US 2005/0209198 A1
(43) Pub. Date: Sep. 22, 2005

(54) Pharmaceutical composition comprising a bisphosphonate and a cox-2 inhibitor for the treatment of bone diseases

(76) Inventors: Zebulun D Horowitz, Basking Ridge, NJ (US); Philip Leonard Simon, Randolph, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/506,039

(22) PCT Filed: Feb. 28, 2003
(86) PCT No.: PCT/EP03/02087

§ 371 Date: Apr. 13, 2005

(30) Foreign Application Priority Data
Feb. 28, 2002 (GB) 0204756.1

(51) Int. Cl. A61K 31/66; A61K 31/415

(52) U.S. Cl. 514/102; 514/406

(57) ABSTRACT

A pharmaceutical composition for treatment of conditions of abnormal bone turnover, comprises in combination a bisphosphonate and a COX-2 inhibitor for simultaneous, sequential or separate use. Also provided is a method of treating a patient suffering from a condition involving abnormal bone turnover, comprising administering to the patient an effective amount of a bis-phosphonates and an effective amount of a COX-2 inhibitor.

This invention relates to bisphosphonates, in particular to new pharmaceuticals uses of bisphosphonates in the treatment of conditions of abnormally increased bone turnover, such as osteoporosis, and compositions containing bisphosphonates for such uses.

Bisphosphonates are widely used to inhibit osteoclast activity in a variety of both benign and malignant diseases, which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity of osteoclasts.

Selective cyclooxygenase inhibitors (COX-2 inhibitors) and their use as non-steroidal anti-inflammatory drugs (NSAIDs) for the treatment of inflammatory disease and pain are well known in the art.

Our copending international patent application WO 01/97788 describes methods for the treatment of conditions of abnormally increased bone turnover in a patient in need of such treatment comprising intermittently administering an effective amount of a bisphosphonate to the patient, wherein the period between administrations is at least about 6 months. The teaching of WO 01/97788 is incorporated by reference in the present description.

It has now been found that surprisingly advantageous results are obtained in the treatment of conditions of abnormally increased bone turnover, such as osteoporosis, if a bisphosphonate is used in combination with a COX-2 inhibitor.

Accordingly the present invention provides a pharmaceutical composition for treatment of a condition involving abnormally increased bone turnover, which comprises in combination a bisphosphonate and a COX-2 inhibitor for simultaneous, sequential or separate use.

Further the invention provides the use of a COX-2 inhibitor for the preparation of a medicament, for use in combination with a bisphosphonate for treatment of a condition involving abnormally increased bone turnover.

In the alternative the invention provides use of a bisphosphonate for the preparation of a medicament for use in combination with a COX-2 inhibitor for treatment of a condition involving abnormally increased bone turnover.

In a further aspect the invention provides a method of treating a patient suffering from a condition involving abnormally increased bone turnover comprising administering to the patient an effective amount of a bisphosphonate and an effective amount of a COX-2 inhibitor.

In yet further aspects the invention provides:
(i) A package comprising a bisphosphonate together with instructions for use in combination with a COX-2 inhibitor for treatment of a condition involving abnormally increased bone turnover, or
(ii) A package comprising a COX-2 inhibitor together with instructions for use in combination with a bisphosphonate for treatment of a condition involving abnormally increased bone turnover.

Conditions of abnormally increased bone turnover which may be treated in accordance with the present invention include: treatment of postmenopausal osteoporosis, e.g. to reduce the risk of osteoporotic fractures; prevention of postmenopausal osteoporosis, e.g. prevention of postmenopausal bone loss; treatment or prevention of male osteoporosis; treatment or prevention of corticosteroid-induced osteoporosis and other forms of bone loss secondary to or due to medication, e.g. diphenylhydantoin, thyroid hormone replacement therapy, treatment or prevention of bone loss associated with immobilisation and space flight; treatment or prevention of bone loss associated with rheumatoid arthritis, osteogenesis imperfecta, hyperthyroidism, anorexia nervosa, organ transplantation, joint prosthesis loosening, and other medical conditions. For example, such other medical conditions may include treatment or prevention of periarticular bone erosions in rheumatoid arthritis; treatment of osteoarthritis, e.g. prevention/treatment of subchondral osteosclerosis, subchondral bone cysts, osteophyte formation, and of osteoarthritic pain, e.g. by reduction in intra-osseous pressure; treatment or prevention of hypercalcemia resulting from excessive bone resorption secondary to hyperparathyroidism, thyrotoxicosis, sarcoidosis or hypervitaminosis D.

Above and elsewhere in the present description the terms “bisphophonate” and “COX-2 inhibitor” include, as appropriate, pharmaceutically acceptable salts and esters thereof.

Thus in the present description the terms “teatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

Advantageously use of a bisphosphonate in combination with a COX-2 inhibitor for treatment of conditions involving abnormally increased bone turnover results in improvement in disease outcome and patient quality of life, in particular in relation to pain management, use of either bisphosphonate or COX-2 inhibitor on their own. Especially there is sustained and long term pain relief, advantageously with early onset of action, after commencement of combined bisphosphonate and COX-2 treatment.

The bisphosphonates for use in the present invention are preferably N-bisphosphonates.

For the purposes of the present description an N-bisphosphonate is a compound which in addition to the characteristic geminal bisphosphate (P—C—P) moiety comprises a nitrogen containing side chain, e.g. a compound of formula I 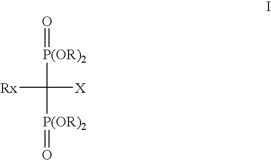
wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles),
   and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II 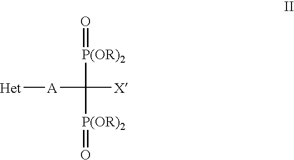
wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a particularly preferred bisphosphonate for use in the invention comprises a compound of Formula III 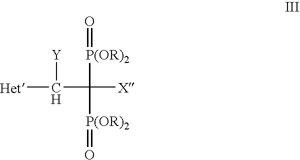
wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a particularly preferred bisphosphonate for use in the invention comprises a compound of Formula IV 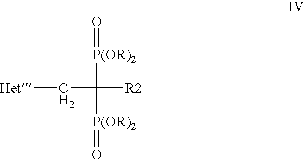
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen, lower radicals having up to and including 7 C-atoms,
   or a pharmacologically acceptable salt thereof.
Examples of particularly preferred N-bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

The most preferred N-bisphosphonate for use in the invention is 2-(imidazol-lyl)-1-hydroxyethane-1,1-diphosphonic acid (zoledronic acid) or a pharmacologically acceptable salt thereof.

All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The N-bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallisation.

The COX-2 inhibitors used in the pharmaceutical compositions and treatment methods of the present invention are typically those which have an IC₅₀ for COX-2 inhibition less than about 2 μM and an IC₅₀ for COX-1 inhibition greater than about 5 μM, e.g. when measured in the assays described by Brideau et al. in Inflamm. Res. 45:68-74 (1996). Preferably the COX-2 inhibitor has a selectivity ratio of at least 10, more preferably at least 40, for COX-2 inhibition over COX-1 inhibition.

Thus, for example, suitable COX-2 inhibitors for use in the invention may include the following compounds or derivatives thereof or a pharmaceutically acceptable salt thereof, or any hydrate thereof: rofecoxib, etoricoxib, celecoxib, valdecoxib, parecoxib, or a 5-alkyl-2-arylaminophenylacetic acid derivative COX-2 inhibitor, e.g. of formula V as defined below.

In a preferred embodiment a COX-2 inhibitor for use in the present invention comprises a compound of formula V 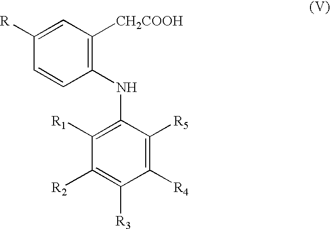
wherein
R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl;
pharmaceutically acceptable salts thereof; and
pharmaceutically acceptable prodrug esters thereof.

Particularly preferred compounds of formula V are those wherein R is methyl or ethyl; R₁ is chloro or fluoro; R₂ is hydrogen; R₃ is hydrogen, fluoro, chloro, methyl or hydroxy; R₄ is hydrogen; and R₅ is chloro, fluoro or methyl; pharmaceutically acceptable salts thereof; and pharmaceutically acceptable esters thereof.

A particularly preferred embodiment relates to the compounds of formula V wherein R is methyl or ethyl; R₁ is fluoro; R₂ is hydrogen; R₃ is hydrogen, fluoro or hydroxy, R₄ is hydrogen; and R₅ is chloro; pharmaceutically acceptable salts thereof; and pharmaceutically acceptable prodrug esters thereof.

Another particularly preferred embodiment of the invention relates to compounds of formula V wherein R is ethyl or methyl; R₁ is fluoro; R₂ is hydrogen or fluoro; R₃ is hydrogen, fluoro, ethoxy or hydroxy, R₄ is hydrogen or fluoro; and R₅ is chloro, fluoro or methyl; pharmaceutically acceptable salts thereof; and pharmaceutically acceptable prodrug esters thereof.

Further are said compounds wherein R is methyl or ethyl; R₁ is fluoro; R₂-R₄ are hydrogen or fluoro; and R₅ is chloro or fluoro; pharmaceutically acceptable salts thereof; and pharmaceutically acceptable prodrug esters thereof.

A further embodiment of the invention relates to the compounds of formula V wherein R is methyl or ethyl; R₁ is fluoro; R₂ is fluoro; R₃ is hydrogen, ethoxy or hydroxy; R₄ is fluoro; and R₅ is fluoro; pharmaceutically acceptable salts thereof; and pharmaceutically acceptable prodrug esters thereof.

Another embodiment of the invention relates to the compounds of formula V wherein R is methyl; R₁ is fluoro; R₂ is hydrogen; R₃ is hydrogen or fluoro; R₄ is hydrogen; and R₅ is chloro; pharmaceutically acceptable salts thereof; and pharmaceutically acceptable prodrug esters thereof.

Particularly preferred embodiments of the invention relate to compounds of formula V
(a) wherein R is methyl; R₁ is fluoro; R₂ is hydrogen; R₃ is hydrogen; R₄ is hydrogen; and R₅ is chloro; pharmaceutically acceptable salts thereof; and pharmaceutically acceptable prodrug esters thereof;
(b) wherein R is methyl; R₁ is fluoro; R₂ is hydrogen; R₃ is fluoro; R₄ is hydrogen; and R₅ is chloro; pharmaceutically acceptable salts thereof; and pharmaceutically acceptable prodrug esters thereof;
(c) wherein R is ethyl; R₁ is fluoro; R₂ is fluoro; R₃ is hydrogen; R₄ is fluoro; and R₅ is fluoro; pharmaceutically acceptable salts thereof; and pharmaceutically acceptable prodrug esters thereof; and
(d) wherein R is ethyl; R₁ is chloro; R₂ is hydrogen; R₃ is chloro; R₄ is hydrogen; and R₅ is methyl; pharmaceutically acceptable salts thereof; and pharmaceutically acceptable prodrug esters thereof.

Pharmaceutically acceptable prodrug esters of the compounds of formula V are ester derivatives which are convertible by solvolysis or under physiological conditions to the free carboxylic acids of formula V. Such esters are e.g. lower ally esters (such as the methyl or ethyl ester), carboxy-lower alkyl esters such as the carboxymethyl ester, nitrooxy-lower alkyl esters (such as the 4-nitrooxybutyl ester), and the like. Preferred prodrugs are the compounds of formula Ia 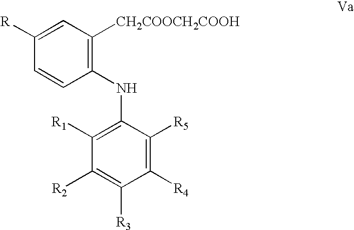
wherein R and R₁-R₅ have meaning as defined hereinabove for compounds of formula V; and pharmaceutically acceptable salts thereof.

Compounds of formula V and Va and their synthesis are described in published international patent applications Nos. WO 99/11605 and WO 01/23346, the teachings of which are incorporated herein by reference.

Pharmacologically acceptable salts of bisphosphonates and COX-2 inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts of the N-bisphosphonates are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts of the N-bisphosphonates is characterized by having one acidic hydrogen and one pharmaceutically acceptable cation, especially sodium, in each of the phosphonic acid groups.

An alternative class of cox-2 inhibitors compounds for use in the invention is the methane sulfonanilide class of inhibitors, of which NS-398, flosulide, nimesulide and (i) are example members.

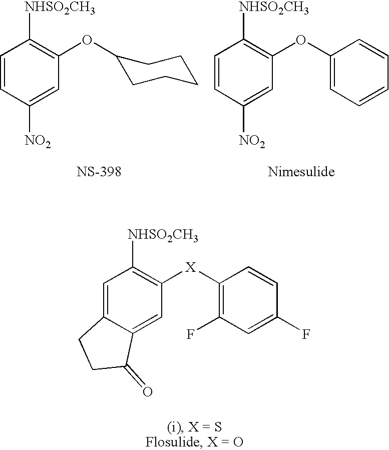

A further class of COX-2 inhibitors is the tricyclic inhibitor class, which can be further divided into the sub-classes of tricyclic inhibitors with a central carbocyclic ring (examples include SC-57666, 1 and 2; those with a central monocyclic heterocyclic ring (examples include DuP 697, SC-58125, SC-58635, SC 236 and 3, 4 and 5); and those with a central bicyclic heterocyclic ring (examples include 6, 7, 8, 9 and 10). Compounds 3, 4, and 5 are described in U.S. Pat. No. 5,474,995.

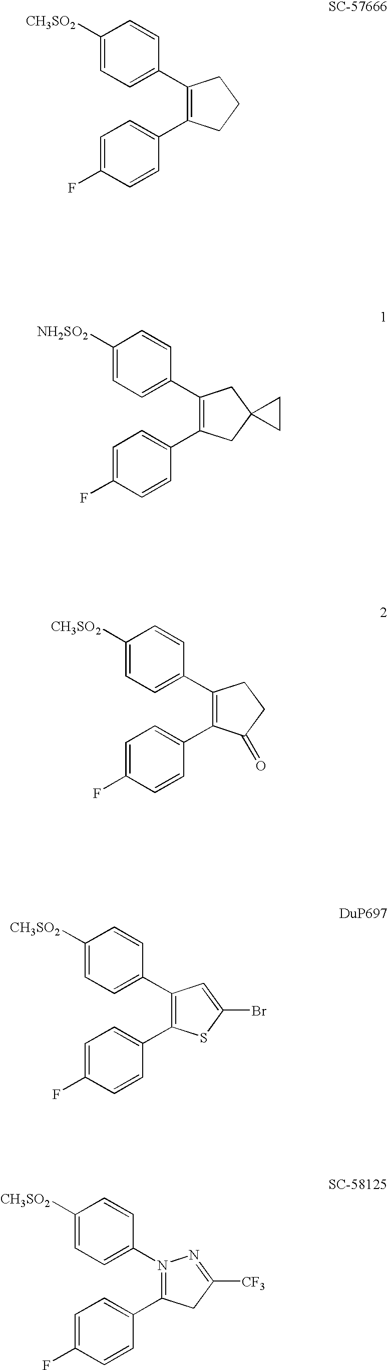

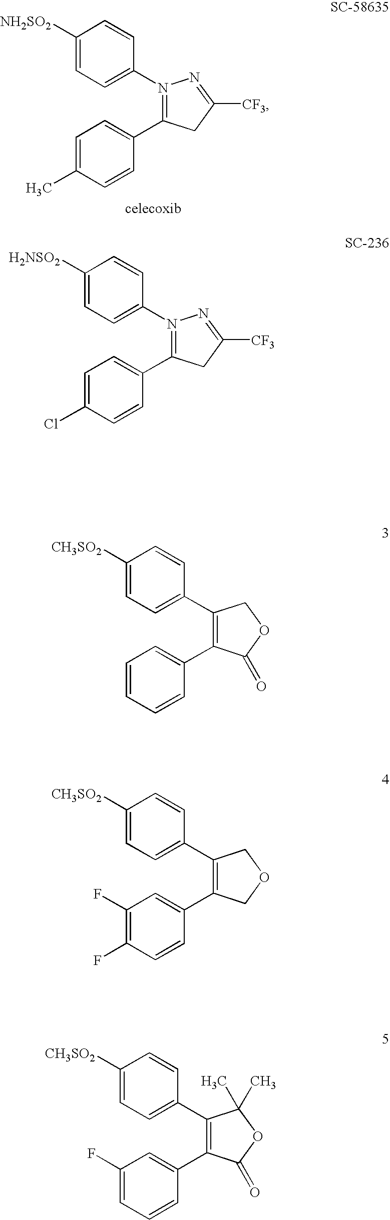

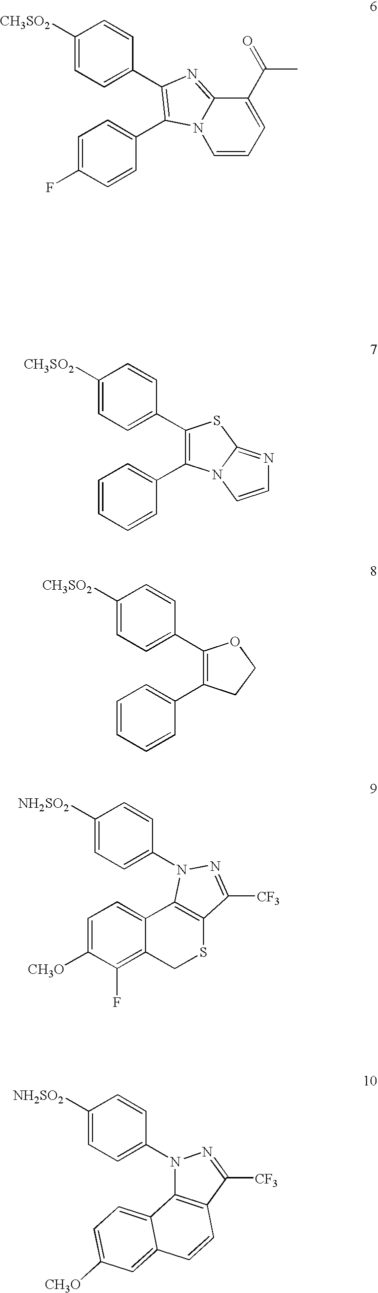

A yet further class of COX-2 inhibitors can be referred to as those which are structurally modified NSAIDS, and includes 11a and structur 11 as example members.

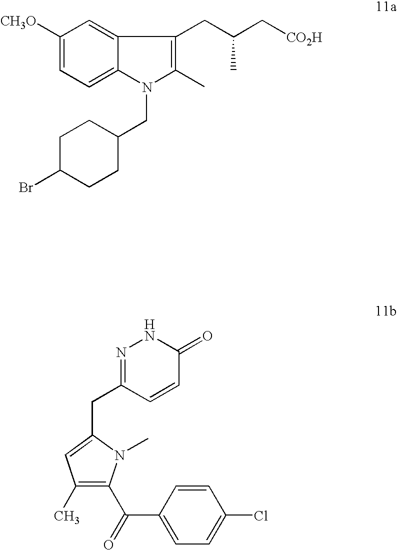

In addition to the structural classes, sub-classes, specific COX-2 inhibitors compound examples, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435.

Additional COX-2 inhibitor compounds which are included in the scope of this invention include.

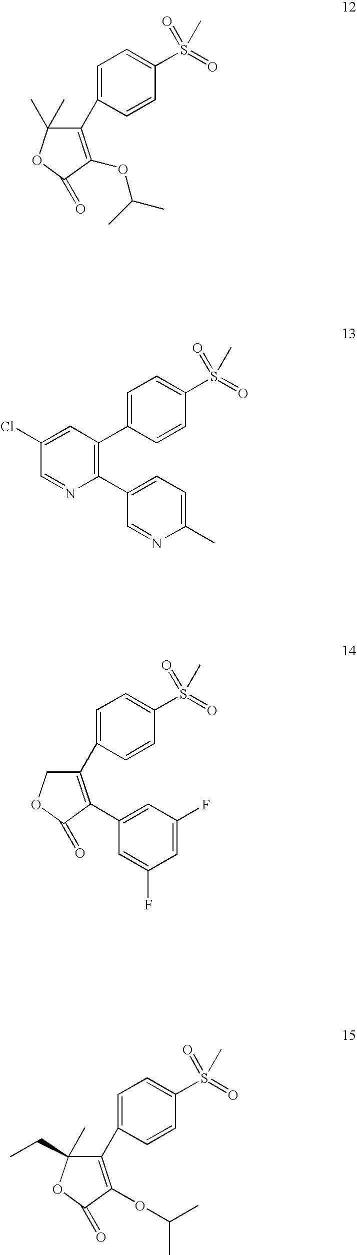

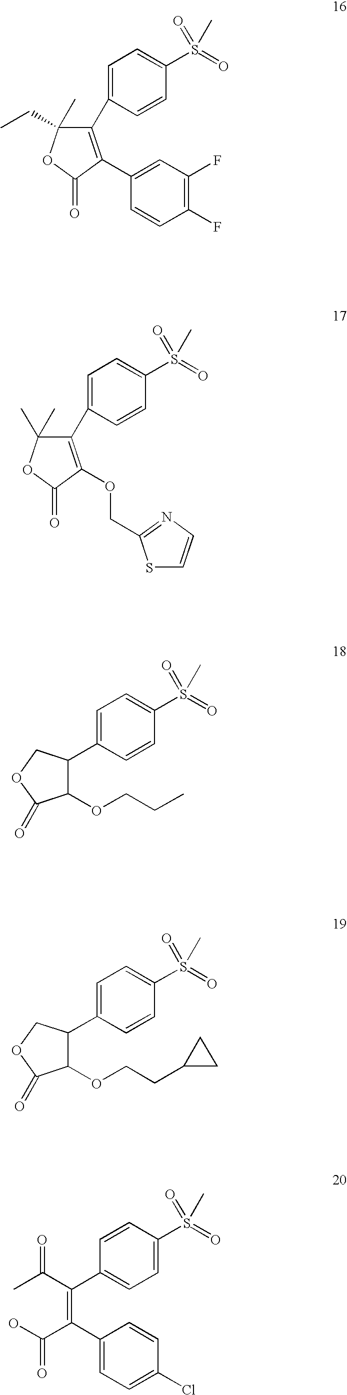

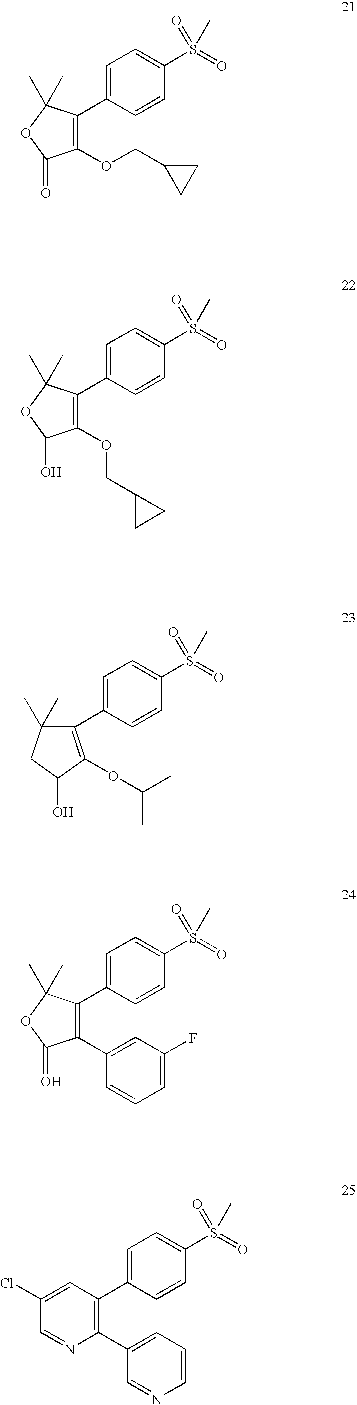

Some of the compounds above can also be identified by the following chemical names:
3: 3-phenyl-4-(4-(methylsulfonyl)phenyl)-2-(5H)-furanone;
4: 3-(3,4-difluorophenyl)-4-(4-(methylsulfonyl)phenyl)-2-(5H)-faranone;
5: 5,5-dimethyl-4-(4-(methylsulfonyl)phenyl)-3-(3-fluorophenyl)-H-furan-2-one;
12: 5,5-dimethyl-4-(4-(methylsulfonyl)phenyl)-3-(2-propoxy)-5H-furan-2-one;
13: 5-chloro-3-(4-(methylsulfonyl)phenyl)-2-(2-methyl-5-pyridinyl)pyridine;
14: 2-(3,5-difluorophenyl)-3-(4-(methylsulfonyl)phenyl)-2-cyclopenten-2-one;
15: 5(S)-5-ethyl-5-methyl-4-(4-methylsulfonyl)phenyl)-3-(2-propoxy)-5H-furan-2-one;
16: 5-ethyl-5-methyl-4-(4-(methylsulfonyl)phenyl)-3-(3,4-difluorophenyl)-5H-furan-2-one;
17: 3-((2-thiazolyl)methoxy)-4-(4-methylsulfonyl)phenyl)-5,5-dymethyl-5H-furan-2-one;
18: 3-propyloxy-4-(4-methylsulfonyl)phenyl)-5,5-dimethyl-5H-furan-2-one;
19: 3-(1-cyclopropylethoxy)-5,5-dimethyl-4-(4-methylsulfonyl)phenyl)-5H-furan-2-one;
20: sodium 2-(4-chlorophenyl)-3-(4-methylsulfonyl)phenyl)-4-oxo-2-pentenoate;
21: 3-(cyclopropylmethoxy)-5,5-dimethyl-4-(4-methylsulfonyl)phenyl)-5H-furan-2-one;
22: 3-(cyclopropylmethoxy)-5,5-dimethyl-4-(4-methylsulfonyl)phenyl)-2,5-dihydrofuran-2-ol;
23: 3-isopropoxy-5,5-dimethyl-4-(4-methylsulfonyl)phenyl)-2,5-dihydrofuran-2-ol;
24: 5,5-dimethyl-3-(3-fluorophenyl)-2-hydroxy-4-(4-methylsulfonyl)phenyl)-2,5-dihydrofuran;
25: 5-Chloro-3-(4-methylsulfonyl)phenyl)-2-(3-pyridinyl)pyridine.

The following publications describe and/or provide methods for making the compounds as indicated: compounds 12, 15, 17, 18, 19 and 21, WO 97/14691; compounds 22, 23 and 24, WO 97/16435; compound 20, WO 96/36623; compound 14, U.S. Pat. No. 5,536,752; compound 16, U.S. Pat. No. 5,474,995. See Examples herein for compounds 13 and 25. Also incorporated herein by reference are those compounds described in WO 96/41645 as having structural Formula VI, shown below, and the definition and preferred definitions and species described therein:

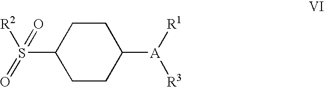

Particulary preferred compounds of formula (VI) include:
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methodoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-(5-(4-chlorophenyl)-3-(trifluoromethyl)-1H-pyrazol-1-yl)benzenesulfonamide;
4-(5-phenyl)-3-(trifluormethyl)-1H-pyrazol-1-yl)benzenesulfonamide;
4-(5-(4-fluorphenyl)-3-(trifluoromethyl)-1H-pyrazol-1-yl)benzenesulfonamide;
4-(5-(4-methoxyphenyl)-3-(trifluormethyl)-1H-pyrazol-1-yl)benzenesulfonamide;
4-(5-(4-chlorophenyl)-3-(difluoromethyl)-1H-pyrazol-1-yl)benzenesulfonamide;
4-(5-(4-methylphenyl)-3-(trifluoromethyl)-1H-pyrazol-1-yl)benzenesulfonamide;
4-(4-chloro-5-(4-chlorohenyl)-3-(trifluoromethyl)-1H-pyrazol-1-yl)benzenesulfonamide;
4-(3-(difluoromethyl)-5-(4-methylphenyl)-1H-pyrazol-1-yl)benzenesulfonamide;
4-(3-(difluoromethyl)-5-phenyl-1H-pyrazol-1-yl)benzenesulfonamide;
4-(3-(difluoromethyl)-5-(4-methoxyphenyl)-1H-pyrazol-1-yl)benzenesulfonamide;
4-(3-cyano-5-(4-fluorophenyl)-1H-pyrazol-1-yl)benzenesulfonamide;
4-(3-(difluoromethyl)-5-(3-fluoro-4-methodoxyphenyl)-1H-pyrazol-1-yl)benzenesulfonamide;
4-(5-(3-fluoro-4-methoxyphenyl)-3-(trifluormethyl)-1H-pyrazol-1-yl)benzenesulfonamide;
4-(4-chloro-5-phenyl-1H-pyrazol-1-yl)benzenesulfonamide;
4-(5-(4-chlorophenyl)-3-hydroxyphenyl)-1H-pyrazol-1-yl)benzenesulfonamide;
4-(5-(N,N-dimethylamino)phenyl)-3-(trifluoromethyl)-1H-pyrazol-1-yl)benzenesulfonamide;
5-(4-fluorophenyl)-6-(4-(methylsulfonyl)phenyl)spiro[2.4]hept-5-ene;
4-(6-(4-fluorophenyl)spiro[2.4]hept-5-en-5-yl)benzenesulfonamide;
6-(4-fluorophenyl)-7-(4-(methylsulfonyl)phenyl)spiro[3.4]oct-6-ene;
5-(3-chloro-4-methoxyphenyl)-6-(4-(methylsulfonyl)phenyl)spiro[2.4]hept-5-ene;
4-(6-(3-chloro-4methoxyphenyl)spiro[2.4]hept-5-en-5-yl)benzenesulfonamide;
5-(3,5-dichloro-4-methodoxyphenyl)-6-(4-(methylsulfonyl)phenyl)spiro[2.4]hept-5-ene;
5-(3-chloro-4-fluorophenyl)-6-(4-(methylsulfonyl)phenyl)spiro[2.4]hept-5-ene;
4-(6-(3,4-dichlorophenyl)spiro[2.4]hept-5-en-5-yl)benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluormethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzenesulfonamide;
4-(4-fluorophenyl)-5-(4-methylsulfonylphenyl)-2-(1-propylamino)thiazole;
2-((3,5-dichlorophenoxy)methyl)-4-(4-fluorophenyl)-5-(4-(methylsulfonyl)phenyl)thiazole;
5-(4-fluorophenyl)-4-(4-methylsulfonylphenyl)-2-trifluoromethylthiazole;
1-methylsulfonyl-4-(1,1-dimethyl-4-(4-fluorophenyl)cyclopenta-2,4-dien-3-yl)benzene;
4-(4-(4-fluorophenyl-1,1-dimethylcyclopenta-2,4-dien-3-yl)benzenesulfonamide;
5-(4-fluorophenyl)-6-(4-(methylsulfonyl)phenyl)spiro[2.4]hepta-4,6-diene;
4-(6-(4-fluorophenyl)spiro[2.4]hepta-4,6-dien-5-yl)benzenesulfonamide;
6-(4-fluorophenyl)-2-methoxy-5-(4-(methylsulfonyl)phenyl)-pyridine-3-carbonitrile;
2-bromo-6-(4-fluorophenyl)-5-(4-(methylsulfonyl)phenyl)-pyridine-3-carbonitrile;
6-(4-fluorophenyl)-5-(4-(methylsulfonyl)phenyl)-2-phenyl-pyridine-3-carbonitrile;
4-(2-(4-methylpyridin-2-yl)-4-(trifluoromethyl)-1H-imidazol-1-yl)benzenesulfonamide;
4-(2-(5-methylpyridin-3-yl)-4-(trifluoromethyl)-1H-imidazol-1-yl)benzenesulfonamide;
4-(2-(2-methylpyridin-3-yl)-4-(trifluoromethyl)-1H-imidazol-1-yl)benzenesulfonamide;
3-(1-(4-(methylsulfonyl)phenyl)-4-(trifluoromethyl)-1H-imidazol-2-yl)benzenesulfonamide;
2-(1-(4-(methylsulfonyl)phenyl)-4-(trifluoromethyl)-1H-imidazol-2-yl)pyridine;
2-methyl-4-(1-(4-(methylsulfonyl)phenyl)-4-(trifluormethyl)-1H-imidazol-2-yl)pyridine;
2-methyl-6-(1-(4-methylsulfonyl)phenyl)-4-(trifluormethyl)-1H-imidazole-2-yl)pyridine;
4-(2-(6-methylpyridin-3-yl)-4-(trifluoromethyl)-1H-imidazol-1-yl)benzenesulfonamide;
2-(3,4-difluorophenyl)-1-(4-(methylsulfonyl)phenyl)-4-(trifluoromethyl)-1H-imidazole;
4-(2-(4-methylphenyl)-4-(trifluoromethyl)-1H-imidazol-1-yl)benzesulfonamide;
2-(4-chlorophenyl)-1-(4-methylsulfonyl)phenyl)-4-methyl-1H-imidazole;
2-(4-chlorophenyl)-1-(4-(methylsulfonyl)phenyl)-4-phenyl-1H-imidazole;
2-(4-chlorophenyl)-4-(4-fluorophenyl)-1-(4-(methylsulfonyl)phenyl)-1H-imidazole;
2-(3-fluoro-4-methoxyphenyl)-1-(4-(methylsulfonyl)phenyl)-4-(trifluoromethyl)-1H-imidazole;
1-(4-methylsulfonyl)phenyl)-2-phenyl-4-trifluoromethyl-1H-imidazole;
2-(4-methylphenyl)-1-(4-(methylsulfonyl)phenyl)-4-trifluoromethyl-1H-imidazole;
4-(2-(3-chloro-4-methylphenyl)-4-(trifluoromethyl-1H-imidazol-1-yl)benzenesulfonamide;
2-(3-fluoro-5-methylphenyl)-1-(4-methylsulfonyl)phenyl)-4-(trifluoromethyl)-1H-imidazole;
4-(2-(3-fluoro-5-methylphenyl)-4-(trifluoromethyl)-1H-imidazol-1-yl)benzenesulfonamide;
2-(3-methylphenyl)-1-(4-(methylsulfonyl)phenyl)-4-(trifluoromethyl)-1H-imidazole;
4-(2-(3-methylphenyl)-4-(trifluoromethyl)-1H-imidazol-1-yl)benzenesulfonamide;
1-(4-(methylsulfonyl)phenyl)-2-(3-chlorophenyl)-4-(trifluoromethyl)-1H-imidazole;
4-(2-(3-chlorophenyl)-4-(trifluoromethyl)-1H-imidazol-1-yl)benzenesulfonamide;
4-(2-phenyl-4-(trifuoromethyl)-1H-imidazol-1-yl)benzenesulfonamide;
4-(2-(4-methodxy-3-chlorophenyl)-4-(trifluoromethyl)-1H-imidazol-1-yl) benzenesulfonamide;
1-allyl-4-(4-fluorophenyl)-3-(4-(methylsulfonyl)phenyl)-5-(trifluoromethyl)-1H-pyrazole;
4-(1-ethyl-4-(4-fluorophenyl)-5-(trifluoromethyl)-1H-pyrazol-3-yl)benzenesulfonamide;
N-phenyl-(4-(4-fluorophenyl)-3-(4-(methylsulfonyl)phenyl)-5-(trifluoromethyl)-H-pyrazol-1-yl)acetamide;
ethyl(4-(4-fluorophenyl)-3-(4-(methylsulfonyl)phenyl)-5-(trifluoromethyl)-1H-pyrazol-1-yl)acetate;
4-(4-fluorophenyl)-3-(4-methylsulfonyl)phenyl)-1-(2-phenylethyl)-1H-pyrazole;
4-(4-fluorophenyl)-3-(4-(methylsulfonyl)phenyl)-1-(2-phenylethyl)-5-(trifluoromethyl)pyrazole;
1-ethyl-4-(4-fluorophenyl)-3-(4-methylsulfonyl)phenyl)-5-(trifluoromethyl)-1H-pyrazole;
5-(4-fluorophenyl)-4-(4-methylsulfonyl)phenyl)-2-(trifluoromethyl)-1H-imidazole;
4-(4-methylsulfonyl)phenyl)-5-(2-thiophenyl)-2-(trifluoromethyl)-1H-imidazole;
5-(4-fluorophenyl)-2-methodoxy-4-(methylsulfonyl)phenyl)-6-(trifluoromethyl)pyridine;
2-ethoxy-5-(4-fluorophenyl)-4-(4-(methylsulfonyl)phenyl)-6-(trifluoromethyl)pyridine;
5-(4-fluorophenyl)-4-(4-(methylsulfonyl)phenyl)-2-(2-propynyloxy)-6-(trifluoromethyl)pyridine;
2-bromo-5-(4-fluorophenyl)-4-(4-methylsulfonyl)phenyl)-6-(trifluoromethyl)pyridine;
4-(2-(3-chloro-4-methoxyphenyl)-4,5-difluorophenyl)benzensulfonamide;
1-(4-fluorophenyl)-2-(4-methylsulfonyl)phenyl)benzene;
5-difluoromethyl-4-(4-methylsulfonyl)phenyl)-3-phenylisoxazole;
4-(3-ethyl-5-phenylisoxazol-4-yl)benzensulfonamid;
4-(5-difluoromethyl-3-phenylisoxazol-4-yl)benzenesulfonamide;
4-(5-hydroxymethyl-3-phenylisoxazol-4-yl)benzenesulfonamide;
4-(5-methyl-3-phenylisoxazol-4-yl)benzenesulfonamide;
1-(2-(4-fluorophenyl)cyclopenten-1-yl)-4-(methylsulfonyl)benzene;
1-(2-(4-fluoro-2-methylphenyl)cyclopenten-1-yl)-4-(methylsulfonyl)benzene;
1-(2-(4-chlorophenyl)cyclopenten-1-yl)-4-(methylsulfonyl)benzene;
1-(2-(2,4-dichlorophenyl)cyclopenten-1-yl)-4-(methylsulfonyl)benzene;
1-(2-(4-trifluoromethylphenyl)cyclopenten-1-yl)-4-(methylsulfonyl)benzene;
1-(2-(4-methylthiophenyl)cyclopenten-1-yl)-4-(methylsulfonyl)benzene;
1-(2-(4-fluorophenyl)-4,4-dimethylcyclopenten-1-yl)-4-(methylsulfonyl)benzene;
4-(2-(4-fluorophenyl)-4,4-dimethylcyclopenten-1-yl)benzesulfonamide;
1-(2-(4-chlorophenyl)-4,4-dimethylcyclopenten-1-yl)-4-(methylsulfonyl)benzene;
4-(2-(4-chlororophenyl)-4,4-dimethylcyclopenten-1-yl)benzenesulfonamide;
4-(2-(4-fluorophenyl)cyclopenten-1-yl)benzenesulfonamide;
4-(2-(4-chlorophenyl)cyclopenten-1-yl)benzenesulfonamide;
1-(2-(4-methoxyphenyl)cyclopenten-1-yl)-4-(methylsulfonyl)benzene;
1-(2-(2,3-difluorophenyl)cyclopenten-1-yl)-4-(methylsulfonyl)benzene;
4-(2-(3-fluoro-4methodyphenyl)cyclopenten-1-yl)benzenesulfonamide;
1-(2-(3-chloro-4-methoxyphenyl)cyclopenten-1-yl)-4-(methylsulfonyl)benzene;
4-(2-(3-chloro-4-fluorophenyl)cyclopenten-1-yl-benzenesulfonamide;
4-(2-(2-methylpyridin-5-yl)cyclopenten-1-yl)benzenesulfonamide;
ethyl 2-(4-(4-fluorophenyl)-5-(4-methylsulfonyl)phenyl)oxazol-2-yl)-2-benzyl-accetate;
2-(4-(4-fluorophenyl)-5-(4-(methylsulfonyl)phenyl)oxazol-2-yl)acetic acid;
2-(tert-butyl)-4-(4-fluorophenyl)-5-(4-methylsulfonyl)phenyl)oxazole;
4-(4-fluorophenyl)-5-(4-(methylsulfonyl)phenyl)-2-phenyloxazole;
4-(4-fluorophenyl)-2-methyl-5-(4-methylsulfonyl)phenyl)oxazole; and
4-(5-(3-fluoro-4-methoxyphenyl)-2-trifluoromethyl-4-oxazolyl)benzenesulfonamide;
   or a pharmaceutically acceptable salt thereof.

The Agents of the Invention, i.e. the COX-2 inhibitor and the bisphosphonate, are preferably used in the form of pharmaceutical preparations that contain the relevant therapeutically effective amount of of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

The particular mode of administration and the dosage may be selected by the attending physician taking into account the particulars of the patient, especially age, weight, life style, activity level and disease state as appropriate. Most preferably, however, the N-bisphosphonate is administered intravenously. Preferably, the bisphosphonate pharmaceutical compositions are adapted to oral or parenteral (especially intravenous, intra-arterial or transdermal) administration. Intravenous and oral, first and foremost intravenous, administration is considered to be of particular importance. Preferably the bisphosphonate active ingredient is in a parenteral form, most preferably an intravenous form.

The dosage of the bisphosphonate for use in the invention may depend on various factors, such as effectiveness and duration of action of the active ingredient, mode of administration, sex, age, weight and individual condition of the patient.

Preferably, the COX-2 pharmaceutical compositions are adapted for oral or parenteral (especially oral) administration. Intravenous and oral, first and foremost oral, adminstration is considered to be of particular importance. Preferably the COX-2 inhibitor active ingredient is in oral form.

The pharmacologically active compounds of the invention are useful in the manufacture of pharmaceutical compositions comprising an effective amount thereof in conjunction or admixture with excipients or carriers suitable for either enteral or parenteral application. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 85%, preferably about 1 to 70%, of the active ingredient.

Tablets may be either film coated or enteric coated according to methods known in the art.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like.

The dosage of COX-2 inhibitor administered is dependent on the species of warm-blooded animal (mammal), the body weight, age and individual condition, and on the form of administration. A unit dosage for oral administration to a mammal of about 50 to 70 kg may contain between about 5 and 1500 mg, e.g. from 100-1000 mg, preferably 200-800 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1500 mg of the active ingredient.

COX-2 inhibitor pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following examples are intended to illustrate the invention and are not to be construed as being limitations thereon.

### EXAMPLES

### A. FORMULATION EXAMPLES

### Example 1

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 percent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)-phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C. The residual water target is 3.5% (with a permissible range of 2.5-4.5%). The dried granulation is passed through a screen using a mill (oscillator) and a 30 mesh screen. The previous steps are repeated to make a second granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature.

Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active |
| fluoroanilino)phenylacetic acid | | substance |
| drug substance | | |
| Microcrystalline cellulose (PH | 51.4 | Filler |
| 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose (PH | 52 | Filler |
| 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1.

### Example 2

An alternative formulation is as set out in Table 3, with information about as percentage W/W, mg/dose, and kg/50,000 tablet batch.

TABLE 3
Alternative formulation composition

| | | | |
|---|---|---|---|
| % w/w | Ingredient | Mg/dose | Kg/batch |

Granulation

| | | | |
|---|---|---|---|
| 65.04 | 5-methyl-2-(2′-chloro-6′-fluoroanilino) | 400.00 | 20.00 |
| | phenylacetic acid drug substance | | |
| 2.15 | Croscarmellose sodium, NF (Ac-Di-Sol) | 13.22 | 0.661 |
| 6.60 | Povidone K30, USP | 40.59 | 2.029 |
| 18.12 | Purified water, USP* | Qs | Qs |

Blending

| | | | |
|---|---|---|---|
| 23.56 | Microcrystalline Cellulose, NF | 144.90 | 6.066 |
| | (Avicel PH 102) | | |
| 2.15 | Croscarmellose sodium, NF (Ac-Di-Sol) | 13.22 | 0.553 |
| 0.50 | Magnesium Stearate, NF (vegetable | 3.07 | 0.128 |
| | source) | | |

Film Coating

| | | | |
|---|---|---|---|
| 84.46 | Opadry, Global White 00F18296 | 15.2028 | 0.296637 |
| 14.03 | Opadry, Global Red 00F15613 | 2.5254 | 0.049275 |
| 1.51 | Opadry, Global Black 00F17713 | 0.2718 | 0.005303 |
| | Purified Water, USP* | Qs | 1.990218 |
| | Film Coated Tablet Weight | 633.00 | |

*Does not appear in final product. Percentage of water added used for granulation based on the dry weight of drug substance and croscarmellose sodium.

The batch is granulated as described in Example 1. The granulation is dried to residual moisture (% LOD) of 1.79%. The formulation process is the same as for the development batches as described above, except for the additional step of coating with Opadry in a coating pan. The coating powders (Opadry) are mixed with purified water to make a 15% w/w coating suspension. The tablets are film coated with the coating suspension in a coating pan using 60° C. to 75° C. inlet air temperature. Based on friability data, a target force of 18 KN (16-20 KN range) is used to compress the remainder of the batch, resulting in acceptable friability (less than 0.5%) and the disintegration times of less than 5 mins. The ejection force is approximately 800 N throughout the compression run. This demonstrates that the blend is lubricated adequately. No picking/sticking is observed on the punch surfaces after 225 minutes. Thus, a smaller size tablet with high drug loading (65%) is achieved using a high shear granulation process, using 17×6.7 mm ovaloid tooling to get tablets with acceptable hardness and friability characteristics.

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1%.

### Example 3

Wet Granulated Tablet Composition

| Amount per tablet | | Ingredient |
|---|---|---|
| 25 | mg | COX-2 inhibitor |
| 79.7 | mg | Microcrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accomodated by varying total weight, and the ratio of the first three ingredients. Generally it is preferable to maintain a 1:1 ratio for microcrystalline cellulose: lactose monohydrate.

### Example 4

Directly Compressed Tablet Composition

| Amount per tablet | | Ingredient |
|---|---|---|
| 25 | mg | COX-2 inhibitor |
| 106.9 | mg | Microcrystalline cellulose |
| 106.9 | mg | Lactose anhydrate |
| 7.5 | mg | Croscarmellose sodium |
| 3.7 | mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### Example 5

Hard Gelatine Capsule Composition

| Amount per capsule | | Ingredient |
|---|---|---|
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystalline cellulose:lactose monohydrate.

### Example 6

Oral Solution

| Amount per 5 mL | | Ingredient |
|---|---|---|
| 50 | mg | COX-2 inhibitor |
| to 5 | mL | with Polyethylene oxide 400 |

### Example 7

Oral Suspension

| Amount per 5 mL dose | | Ingredient |
|---|---|---|
| 101 | mg | COX-2 inhibitor |
| 150 | mg | Polyvinylpyrrolidone |

Oral Suspension

| Amount per 5 mL dose | | Ingredient |
|---|---|---|
| 2.5 | mg | Poly oxyethylene sorbitan monolaurate |
| 10 | mg | Benzoic acid |
| to 5 | mL | with sorbitol solution (70%) |

Suspension dose strengths of between 1 and 50 mg/5 ml can be accomodated by varying the ratio of the first two ingredients.

### Example 8

Intravenous Infusion

| Amount per 200 mL dose | | Ingredient |
|---|---|---|
| 1 | mg | COX-2 inhibitor |
| 0.2 | mg | Polyethylene oxide 400 |
| 1.8 | mg | Sodium chloride |
| to 200 | mL | Purified water |

### Example 9

Capsules containing coated pellets of active ingredient, for example, disodium pamidronate pentahydrate, as active ingredient:

| Core pellet: | | |
|---|---|---|
| active ingredient (ground) | 197.3 | mg |
| Microcrystalline cellulose | 52.7 | mg |
| (Avicel ® PH 105) | 250.0 | mg |
| +Inner coating: | | |
| Cellulose HP-M 603 | 10.0 | mg |
| Polyethylene glycol | 2.0 | mg |
| Talc | 8.0 | mg |
| | 270.0 | mg |
| +Gastric juice-resistant outer coating: | | |
| Eudragit ® L 30 D (solid) | 90.0 | mg |
| Triethyl citrate | 21.0 | mg |
| Antifoam ® AF | 2.0 | mg |
| Water | | |
| Talc | 7.0 | mg |
| | 390.0 | mg |

A mixture of disodium pamidronate with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 10

Monolith adhesive transdermal system, containing as active ingredient, for example, 1-hydroxy-2-(imidazol-1-yl)-ethane-1,1-diphosphonic acid:

Composition:

| | | |
|---|---|---|
| polyisobutylene (PIB) 300 | 5.0 | g |
| (Oppanol B1, BASF) | | |
| PIB 35000 | 3.0 | g |
| (Oppanol B10, BASF) | | |
| PIB 1200000 | 9.0 | g |
| (Oppanol B100, BASF) | | |
| hydrogenated hydrocarbon resin | 43.0 | g |
| (Escorez 5320, Exxon) | | |
| 1-dodecylazacycloheptan-2-one | 20.0 | g |
| (Azone, Nelson Res., Irvine/CA) | | |
| active ingredient | 20.0 | g |
| Total | 100.0 | g |

Preparation:

The above components are together dissolved in 150 g of special boiling point petroleum fraction 100-125 by rolling on a roller gear bed. The solution is applied to a polyester film (Hostaphan, Kalle) by means of a spreading device using a 300 mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 11

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

Composition:

| | | |
|---|---|---|
| active ingredient (free diphosphonic acid) | 1.0 | mg |
| mannitol | 46.0 | mg |
| Trisodium citrate × 2 H₂O ca. | 3.0 | mg |
| water | 1 | ml |
| water for injection | 1 | ml. |

In 1 ml of water, the active ingredient is titrated with trisodium citrate×2H₂O to pH 6.0. Then, the mannitol is added and the solution is lyophilized and the lyophilisate filled into a vial.

### Example 12

Ampoule containing active ingredient, for instance disodium pamidronate pentahydrate dissolved in water. The solution (concentration 3 mg/ml) is for i.v. infusion after dilution.

Composition:

| | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Example 13

### Treatment of Patients

“A Multinational, Randomized, Double-Blind, Placebo-Controlled, Parallel-Group, Dose-Ranging, Safety and Efficacy Trial With Intravenous Bolus Injections of Zoledronate In the Treatment of Postmenopausal Osteoporosis”.

A dose and dose regimen-finding 24 months trial of iv zoledronic acid in patients with postmenopausal osteoporosis is carried out. Three hundred and fifty one patients are randomized to six study arms. Patients who have recent exposure to bone active drugs, e.g. bisphosphonates, estrogen, calcitonin, raloxifene, or a history of metabolic bone diseases are excluded. All patients are evaluated at baseline and in 3-monthly visits. Zoledronic acid or placebo was administered as a bolus iv injection into a peripheral vein over 5 minutes at every visit. Patients from both zoledronic acid treated and placebo groups also receive an oral COX-2 inhibitor (5-methyl-2-(2′-chloro-6′-fluoroanilino)phenylacetic acid—400 mg per day p.o.) or oral placebo.

Efficacy is ascertained by measurement of percent change from baseline in bone mineral density (BMD) measured by dual energy X-ray absorptiometry (DEXA) as compared to placebo, at 6, 9, and 12 months. Patients also maintain records of pain scores on a once weekly basis and are monitored for pain scores at the 3-monthly visits.

As a special safety measure trans-iliac bone biopsies are obtained in a subset of patients from all study arms at 12 months, and X-rays of the thoracic and lumbar spine from all study participants are evaluated at baseline and at 12 months for the occurrence of incident vertebral fractures.

Additionally, the degree and duration of suppression of biochemical markers of bone turnover—parathyroid hormone (PTH), bone specific alkaline phosphatase (BSAP), serum C-telopeptide (CTX), serum osteocalcin, urine N-telopeptide (NTX)/creatinine ratio, urine deoxypyridinoline (d-pyd)/creatinine ratio, urine pyridinoline (pyd)/creatinine ratio—is obtained every 3 months and measured in a central laboratory.

### Study Arms

Placebo
0.25 mg zoledronic acid every 3 months
0.5 mg zoledronic acid every 3 months
1.0 mg zoledronic acid every 3 months
2.0 mg zoledronic acid every 6 months
4.0 mg zoledronic acid every 12 months
   each with 50% COX-2 treatment and 50% oral placebo.

The BMD data indicate that zoledronic acid dose administration as infrequent as every 6 or 12 months can safely result in a statistically significant and medically relevant bone mass increase. It is believed that these data further indicate that a continued preservation of new bone beyond one year, without additional dose administration, is likely or that further bone mass increase is possible. It is also believed that re-treatment in additional cycles of every 6 month, 12 month, or less frequent dose administration will lead to further BMD increase. A reduction of risk of osteoporotic fracture is expected to accompany the bone mass increases.

In addition the pain scores and general well being of patients in the COX-2 treated groups show improvement over the oral placebo treated groups.

1. A pharmaceutical composition for treatment of a condition involving abnormally increased bone turnover which comprises in combination a bisphosphonate and a COX-2 inhibitor for simultaneous, sequential or separate use.

2. A method of treating a patient suffering from a condition involving abnormally increased bone turnover comprising administering to the patient an effective amount of a bisphosphonate and an effective amount of a COX-2 inhibitor.

3. (canceled)

4. (canceled)

5. A package comprising a bisphosphonate together with instructions for use in combination with a COX-2 inhibitor for treatment of a condition involving abnormally increased bone turnover.

6. The composition according to claim 1 in which the bisphosphonate is an N-bisphosphonate.

7. A composition according to claim 6 in which the bisphosphonate is a compound of formula I 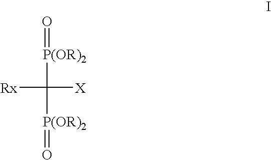
wherein
X is hydrogen, hydroxyl, amino, alkanoyl, or an amino group substituted by C-C4 alkyl, or alkanoyl;
R is hydrogen or C-C4 alkyl and
Rx is a side chain which contains an optionally substituted amino group, or a nitrogen containing heterocycle (including aromatic nitrogen-containing heterocycles), or a pharmaceutically acceptable salt thereof or any hydrate thereof.

8. A composition according to claim 7, in which the bisphosphonate is 2-1(imidazol-lyl)-I-hydroxyethane-I,I-diphosphonic acid (zoledronic acid) or a pharmacologically acceptable salt thereof

9. A composition according to claim 1 in which the COX-2 inhibitor is a COX-2 inhibitor which has an IC50 for COX-2 inhibition less than about 2 pM and an ICso for COX-1 inhibition greater than about 5,uM.

10. A composition according to claim 8 in which the COX-2 inhibitor is selected from the group consisting of rofecoxib, etoricoxib, celecoxib, valdecoxib, parecoxib, or a 5-alkyl-2-arylaminophenylacetic acid derivative COX-2 inhibitor, or a pharmaceutically acceptable salt thereof, or any hydrate thereof.

11. A package comprising a COX-2 inhibitor together with instructions for use in combination with a bisphosphonate for treatment of a condition involving abnormally increased bone turnover.

12. A method according to claim 2 in which the bisphosphonate is an N-bisphosphonate.

13. A method according to claim 2 in which the COX-2 inhibitor is a COX-2 inhibitor which has an IC50 for COX-2 inhibition less than about 2 pM and an ICso for COX-1 inhibition greater than about 5,uM.

14. A method according to claim 12 in which the bisphosphonate is a compound of formula I 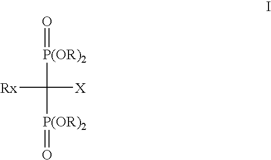
wherein
X is hydrogen, hydroxyl, amino, alkanoyl, or an amino group substituted by C-C4 alkyl, or alkanoyl;
R is hydrogen or C-C4 alkyl and
Rx is a side chain which contains an optionally substituted amino group, or a nitrogen containing heterocycle (including aromatic nitrogen-containing heterocycles), or a pharmaceutically acceptable salt thereof or any hydrate thereof.

15. A method according to claim 14, in which the bisphosphonate is 2-1 (imidazol-lyl)-I-hydroxyethane-I,I-diphosphonic acid (zoledronic acid) or a pharmacologically acceptable salt thereof

16. A method according to claim 15 in which the COX-2 inhibitor is selected from the group consisting of rofecoxib, etoricoxib, celecoxib, valdecoxib, parecoxib, or a 5-alkyl-2-arylaminophenylacetic acid derivative COX-2 inhibitor, or a pharmaceutically acceptable salt thereof, or any hydrate thereof
